(19) United States
(12) Patent Application Publication
(10) Pub. No.: US 2025/0302404 A1
(43) Pub. Date: Oct. 2, 2025

(54) METHODS AND APPARATUS FOR DEEP LEARNING BASED MOTION DETECTION IN NUCLEAR IMAGING SYSTEMS

(71) Applicant: Siemens Medical Solutions USA, Inc., Malvern, PA (US)

(72) Inventors: Joshua Schaefferkoetter, Knoxville, TN (US); Maurizio Conti, Knoxville, TN (US)

(21) Appl. No.: 18/618,372

(22) Filed: Mar. 27, 2024

(51) Int. Cl. A61B 6/03; G06F 3/14; G06V 10/44; G06V 10/82; G16H 30/40

(52) U.S. Cl. CPC A61B 6/037; A61B 6/032; G06F 3/14; G06V 10/44; G06V 10/82; G16H 30/40

(57) ABSTRACT

Systems and methods for detecting subject motion within medical images based on trained deep learning processes are disclosed. In some examples, an image processing system receives positron emission tomography (PET) measurement data and co-modality measurement data from an image scanner. The image processing system generates PET images and co-modality images based on the PET measurement data and co-modality measurement data, respectively. Further, the image processing system inputs the PET images and the co-modality images to a first trained neural network, and generates first features of the PET measurement data and second features of the co-modality measurement data. The image processing system inputs the first features and the second features to a second trained neural network and, generates displacement data characterizing a displacement between the first features and the second features. Based on the displacement data, the image processing system generates display data for display.

### FIELD

Aspects of the present disclosure relate in general to medical diagnostic systems and, more particularly, to detecting subject motion in nuclear images for diagnostic and reporting purposes.

### BACKGROUND

Nuclear imaging systems can employ various technologies to capture images. For example, some nuclear imaging systems employ positron emission tomography (PET) or single-photon emission computed tomography (SPECT) to capture anatomical images. PET is nuclear medicine imaging techniques that produces tomographic images representing the distribution of positron emitting isotopes within a body, while SPECT relies on the detection of gamma rays to produce tomographic images representing the distribution of radioactive tracer molecules within a body. Some nuclear imaging systems employ a co-modality, such as computed tomography (CT) or Magnetic Resonance Imaging (MRI). CT is an imaging technique that uses x-rays to produce anatomical images. Magnetic Resonance Imaging (MRI) is an imaging technique that uses magnetic fields and radio waves to generate anatomical and functional images. Some nuclear imaging systems combine images from PET and CT scanners during an image fusion process to produce images that show information from both a PET scan and a CT scan (e.g., PET/CT systems). Similarly, some nuclear imaging systems combine images from PET and MRI scanners to produce images that show information from both a PET scan and an MRI scan.

Typically, these nuclear imaging systems capture measurement data, and process the captured measurement data using mathematical algorithms to reconstruct medical images. For example, reconstruction can be based on models based on analytic or iterative algorithms or, more recently, deep learning algorithms. In at least some instances, conventional image reconstruction (e.g., PET image reconstruction) and clinical interpretation assume, and may depend on, spatial consistency between various modality scans. For instance, reconstruction and/or clinical interpretation of PET and CT scans may assume that the PET and CT scans are spatially consistent. During image capture, however, subjects (e.g., patients) may move. For instance, a subject may intentionally or unintentionally move their head, arm, or leg. As another example, a subject may move due to breathing. These movements may cause a misalignment of the PET and CT images. For example, tissues captured in the PET image may not align with tissues captured in the CT image. As a result, the movement may cause errors during the reconstruction process, and may further hinder clinical interpretation of the reconstructed image, among other potential problems. As such, there are opportunities to address deficiencies in nuclear imaging systems.

### SUMMARY

Systems and methods for detecting subject motion within medical images based on trained deep learning processes are disclosed.

In some embodiments, a non-transitory computer readable medium stores instructions that, when executed by at least one processor, cause the at least one processor to perform operations including receiving positron emission tomography (PET) measurement data and co-modality measurement data from an image scanning system. The operations also include generating a PET image based on the PET measurement data and a co-modality image based on the co-modality measurement data. Further, the operations include inputting the PET image and the co-modality image to a first trained neural network and, based on inputting the PET image and the co-modality image to the first trained neural network, generating first features of the PET image and second features of the co-modality image. The operations also include inputting the first features and the second features to a second trained neural network and, based on inputting the first output data to the second trained neural network, generating displacement data characterizing a displacement between the first features and the second features. The operations further include generating display data based on the displacement data, and transmitting the display data for display.

In some embodiments, a system includes a memory device storing instructions and at least one processor communicatively coupled the memory device. The at least one processor is configured to execute the instructions to receive positron emission tomography (PET) measurement data and co-modality measurement data from an image scanning system. The at least one processor is also configured to execute the instructions to generate a PET image based on the PET measurement data and a co-modality image based on the co-modality measurement data. Further, the at least one processor is configured to execute the instructions to input the PET image and the co-modality image to a first trained neural network and, based on inputting the PET image and the co-modality image to the first trained neural network, generate first features of the PET image and second features of the co-modality image. The at least one processor is also configured to execute the instructions to input the first features and the second features to a second trained neural network and, based on inputting the first output data to the second trained neural network, generate displacement data characterizing a displacement between the first features and the second features. The at least one processor is further configured to execute the instructions to generate display data based on the displacement data, and transmit the display data for display.

In some embodiments, a computer-implemented method includes receiving positron emission tomography (PET) measurement data. The method also includes receiving co-modality measurement data. Further, the method includes inputting the PET measurement data and the co-modality measurement data to a neural network and, based on inputting the PET measurement data and the co-modality data to the neural network, generating output data characterizing first features of the PET measurement data and second features of the co-modality measurement data. The method also includes determining the neural network is trained based on the output data. Based on the determination, the method further includes storing parameters characterizing the neural network in a data repository.

In some embodiments, a non-transitory computer readable medium stores instructions that, when executed by at least one processor, cause the at least one processor to perform operations including receiving positron emission tomography (PET) measurement data. The operations also include receiving co-modality measurement data. Further, the operations include inputting the PET measurement data and the co-modality measurement data to a neural network and, based on inputting the PET measurement data and the co-modality data to the neural network, generating output data characterizing first features of the PET measurement data and second features of the co-modality measurement data. The operations also include determining the neural network is trained based on the output data. Based on the determination, the operations further include storing parameters characterizing the neural network in a data repository.

In some embodiments, a system includes a memory device storing instructions and at least one processor communicatively coupled the memory device. The at least one processor is configured to execute the instructions to receive positron emission tomography (PET) measurement data. The at least one processor is also configured to execute the instructions to receive co-modality measurement data. Further, the at least one processor is configured to execute the instructions to input the PET measurement data and the co-modality measurement data to a neural network and, based on inputting the PET measurement data and the co-modality data to the neural network, generate output data characterizing first features of the PET measurement data and second features of the co-modality measurement data. The at least one processor is also configured to execute the instructions to determine the neural network is trained based on the output data. Based on the determination, the at least one processor is further configured to execute the instructions to store parameters characterizing the neural network in a data repository

In some embodiments, a computer-implemented method includes receiving first features generated from positron emission tomography (PET) measurement data and second features generated from co-modality measurement data. The method also includes inputting the first features and the second features to a neural network and, based on the inputting the first features and the second features to the neural network, generating output data characterizing a displacement between the first features and the second features. Further, the method includes determining the neural network is trained based on the output data. Based on the determination, the method further includes storing parameters characterizing the neural network in a data repository.

In some embodiments, a non-transitory computer readable medium stores instructions that, when executed by at least one processor, cause the at least one processor to perform operations including receiving first features generated from positron emission tomography (PET) measurement data and second features generated from co-modality measurement data. The operations also include inputting the first features and the second features to a neural network and, based on the inputting the first features and the second features to the neural network, generating output data characterizing a displacement between the first features and the second features. Further, the operations include determining the neural network is trained based on the output data. Based on the determination, the operations further include storing parameters characterizing the neural network in a data repository.

In some embodiments, a system includes a memory device storing instructions and at least one processor communicatively coupled the memory device. The at least one processor is configured to execute the instructions to receive first features generated from positron emission tomography (PET) measurement data and second features generated from co-modality measurement data. The at least one processor is also configured to execute the instructions to input the first features and the second features to a neural network and, based on the inputting the first features and the second features to the neural network, generate output data characterizing a displacement between the first features and the second features. Further, the at least one processor is configured to execute the instructions to determine the neural network is trained based on the output data. Based on the determination, the at least one processor is further configured to execute the instructions to store parameters characterizing the neural network in a data repository.

In some embodiments, a computer-implemented method includes receiving positron emission tomography (PET) measurement data and co-modality measurement data from an image scanning system. The method also includes generating a PET image based on the PET measurement data and a co-modality image based on the co-modality measurement data. Further, the method includes inputting the PET image and the co-modality image to a first trained neural network and, based on inputting the PET image and the co-modality image to the first trained neural network, generating first features of the PET image and second features of the co-modality image. The method also includes inputting the first features and the second features to a second trained neural network and, based on inputting the first output data to the second trained neural network, generating displacement data characterizing a displacement between the first features and the second features. The method further includes generating display data based on the displacement data, and transmitting the display data for display.

### BRIEF DESCRIPTION OF THE DRAWINGS

The following will be apparent from elements of the figures, which are provided for illustrative purposes and are not necessarily drawn to scale.

FIG. 1 illustrates a nuclear image system, in accordance with some embodiments.

FIG. 2 illustrates a block diagram of an example computing device that can perform one or more of the functions described herein, in accordance with some embodiments.

FIGS. 3A and 3B illustrate exemplary display data generated by the nuclear image system of FIG. 1, in accordance with some embodiments.

FIG. 4A illustrates an image reconstructed from misaligned Positron Emission Tomography (PET) and computed tomography (CT) image data.

FIG. 4B illustrates an image characterizing displacement vector values for the image of FIG. 4A, in accordance with some embodiments.

FIG. 4C illustrates a heat map generated based on the displacement vector values of FIG. 4B, in accordance with some embodiments.

FIG. 5 illustrates the training of neural networks in a nuclear imaging system, in accordance with some embodiments.

FIG. 6 is a flowchart of an example method to train neural networks, in accordance with some embodiments.

FIG. 7 is a flowchart of an example method to detect subject motion using trained neural networks, in accordance with some embodiments.

### DETAILED DESCRIPTION

This description of the exemplary embodiments is intended to be read in connection with the accompanying drawings, which are to be considered part of the entire written description.

The exemplary embodiments are described with respect to the claimed systems as well as with respect to the claimed methods. Furthermore, the exemplary embodiments are described with respect to methods and systems for image reconstruction, as well as with respect to methods and systems for training functions used for image reconstruction. Features, advantages, or alternative embodiments herein can be assigned to the other claimed objects and vice versa. For example, claims for the providing systems can be improved with features described or claimed in the context of the methods, and vice versa. In addition, the functional features of described or claimed methods are embodied by objective units of a providing system. Similarly, claims for methods and systems for training image reconstruction functions can be improved with features described or claimed in context of the methods and systems for image reconstruction, and vice versa.

Various embodiments of the present disclosure can employ machine learning methods or processes to provide clinical information from nuclear imaging systems. For example, the embodiments can employ machine learning methods or processes to reconstruct images based on captured measurement data, and provide the reconstructed images for clinical diagnosis. In some embodiments, machine learning methods or processes are trained to improve the reconstruction of images and clinical interpretations of those reconstructed images.

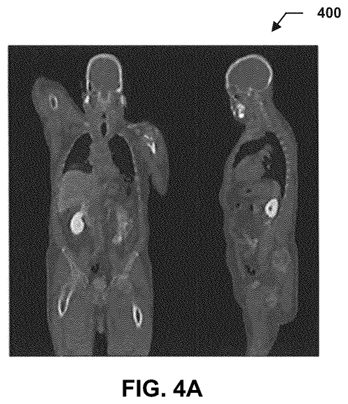

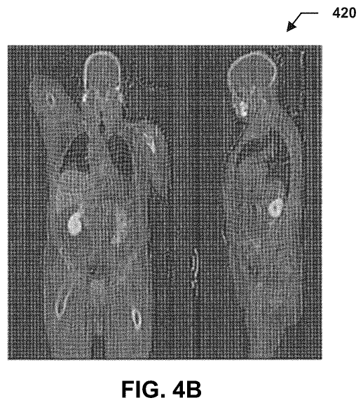

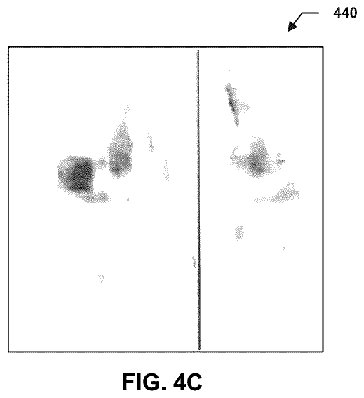

Hybrid imaging systems, such as PET/CT imaging systems, can provide two independent modalities. For example, a PET/CT imaging system can capture PET scans of a subject as well as CT scans of the subject. If a subject moves during or between these image captures, the PET and CT images may be misaligned (e.g., tissue located at a position in a PET image may be located at another position in the CT image). FIG. 4A, for example, illustrates a display of a mis-registered PET/CT image 400 (e.g., a PET image misaligned with a CT image). The PET/CT image 400 includes a coronal view and a sagittal view of a scanned subject, and are orthogonal two-dimensional (2D) slices from a same 3D volume. As illustrated, there is a visible misalignment between the PET and CT images due to respiration. For example, there is a mismatch at the boundary between the lung and liver. Indeed, some of the liver activity on the PET image appears to sit within the space of the lung on the CT image.

The embodiments described herein may detect (e.g., determine and generate an estimate of) such inter-modal movement and, in some examples, may display an indication of the inter-modal movement. For instance, the embodiments may include a deep learning motion estimation framework that includes at least two trained artificial intelligence or machine learning models, such as neural networks (e.g., convolutional neural networks (CNNs). The first trained neural network can processes an image from each modality (e.g., normalized images) to generate features corresponding to each modality. The second trained neural network processes the generated features from the first trained neural network, and generates displacement data characterizing a relative motion displacement between the features generated from each modality. The displacement data may include 3-dimensional (3D) displacement values for each of a plurality of pixel positions, for example.

The displacement data may be utilized in various ways to assist a medical professional (e.g., a physician) during clinical interpretation of the images. For example, a graphical display, such as a heat map, may be generated based on the displacement data. The heat map may indicate displacement magnitudes (e.g., Euclidian magnitudes) for each of a plurality of voxels. The heat map may be displayed to a medical professional to assist in clinical interpretation of a fused image reconstructed from the images for each modality. In some examples, an alert (e.g., a warning icon for display) may be generated based on the displacement data. For example, the alert may be generated when a displacement magnitude is beyond a corresponding threshold. The alert may warn the medical professional that a relatively large movement was detected between the various modality image scans.

As described herein, the first neural network can be trained based on training data that includes measurement data for each modality. For example, the first neural network may be trained based on training data that includes PET measurement data and corresponding co-modality measurement data (e.g., CT measurement data that corresponds to PET scans of a same subject). In some examples, the PET measurement data and/or co-modality measurement data is labelled to identify various features (e.g., anatomical regions, tissues, etc.). During training, the first neural network generates output data characterizing first features of the PET measurement data and second features of the co-modality measurement data. For instance, the first neural network may perform various linear (e.g., convolutional) and nonlinear operations and, as a result, output the first features comprising a set of learned spatial features. The first features and second features may include common features detected in each of the PET measurement data and co-modality measurement data.

A determination as to whether the first neural network is sufficiently is trained can be made based on the output data. For instance, a metric value may be computed based on the output data and expected feature data characterizing expected feature detections. The metric value may be, for instance, a metric value computed from a loss function, such as computed precision values, computed recall values, a computed AUC value, any receiver operating characteristic (ROC) curve or precision-recall (PR) curve value, or any other suitable metric value. The determination as to whether the first neural network is trained can be made based on the metric value. For instance, the first neural network may be considered trained when the computed metric value is beyond (e.g., below, above) a corresponding threshold.

The second neural network can be trained based on training data that includes first features of the PET measurement data and corresponding second features of the co-modality measurement data (e.g., CT measurement data). The training data may be labelled to indicate displacements between corresponding pixels of the first features and the second features. During training, the second neural network generates output data characterizing displacements between the corresponding pixels of the first features and the second features. A determination as to whether the second neural network is sufficiently is trained can be made based on the output data. For instance, the displacements (i.e., displacement values) provided by the output data may be compared to expected displacements to determine whether the second neural network is sufficiently trained.

In some examples, a metric value is computed based on the output data and expected displacement values, and the determination as to whether the second neural network is trained is made based on the metric value. The metric value may be, for instance, a metric value computed from a loss function, such as computed precision values, computed recall values, a computed AUC value, any ROC curve or PR curve value, or any other suitable metric value. The second neural network may be considered trained when the computed metric value is beyond (e.g., below, above) a corresponding threshold.

In some examples, to generate the training data for the second neural network, one or more of the first features of the PET measurement data and/or the second features of the co-modality measurement data may be adjusted to introduce a displacement between corresponding features (e.g., tissues). Alternatively, the PET measurement data and/or the co-modality measurement data may be adjusted to introduce the displacements, and the first features and second features may then be generated based on inputting the adjusted PET measurement data and/or the co-modality measurement data to the trained first neural network. The training data may be labelled to indicate the displacements between corresponding pixels of the first features and the second features. For instance, the training data may include 3D vectors that include displacement offsets in three dimensions (e.g., x, y, and z directions of the x, y, z coordinate system) for at least some of the pixels of the first features and the second features.

Among other advantages, the embodiments may provide medical professionals with an automatic quality control feature to assist with clinical interpretation of images. Moreover, the embodiments may provide an indication that the captured PET and CT images are not spatially consistent, thereby allowing a patient to be rescanned if necessary (e.g., such as when the PET and CT images are misaligned by at least a threshold amount, as described herein) before the patient leaves the imaging room.

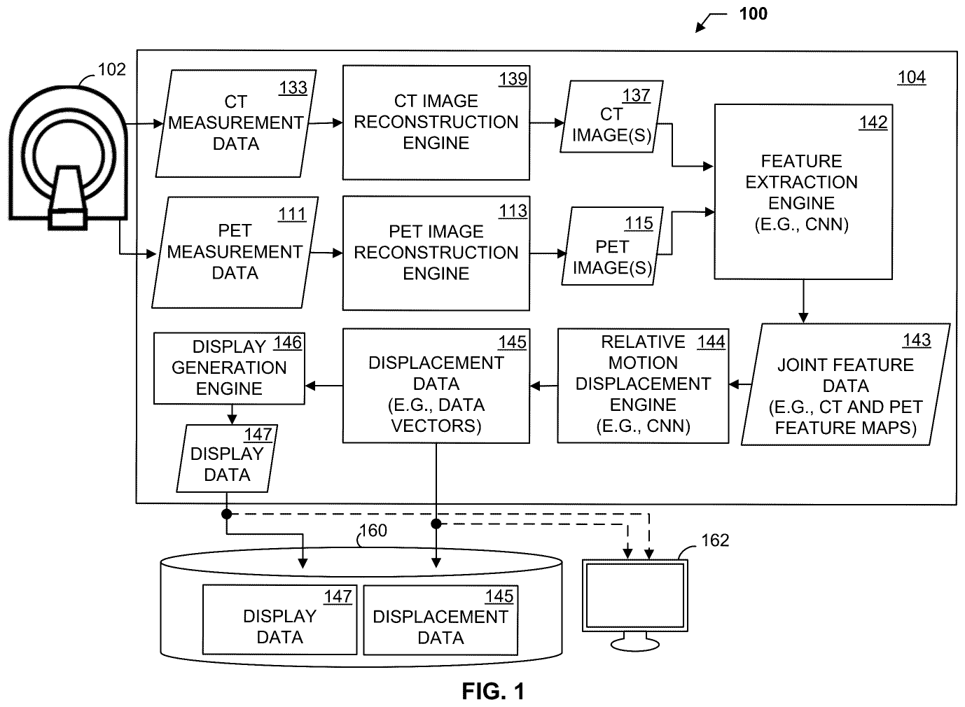

FIG. 1 illustrates an exemplary nuclear imaging system 100. As illustrated, nuclear imaging system 100 includes image scanning system 102, image processing system 104, data repository 160, and, in some examples, monitor 162. Image scanning system 102 can be, for example, a PET/CT scanner that can capture PET and CT images. For instance, image scanning system 102 can capture CT images of anything in a CT's scanner field of view (FOV) (e.g., of a person), and generate CT measurement data 133 based on the CT scans. Image scanning system 102 can also capture PET images (e.g., of the person) of anything in the PET's scanner FOV, and generate PET measurement data 111 (e.g., sinogram data) based on the captured PET images. The PET measurement data 111 can represent anything imaged in the scanner's FOV that contains positron emitting isotopes. In at least some examples, the CT measurement data 133 and the PET measurement data 111 correspond to the scanning of a same subject (e.g., patient). Image scanning system 102 can transmit the CT measurement data 133 and the PET measurement data 111 to image processing system 104.

Image processing system 104 includes CT image reconstruction engine 139, PET image reconstruction engine 113, feature extraction engine 142, relative motion displacement engine 144, and display generation engine 146. In some examples, all or parts of image processing system 104 are implemented in hardware, such as in one or more field-programmable gate arrays (FPGAs), one or more application-specific integrated circuits (ASICs), one or more state machines, one or more computing devices, digital circuitry, or any other suitable circuitry. In some examples, parts or all of image processing system 104 can be implemented in software as executable instructions such that, when executed by one or more processors, cause the one or more processors to perform respective functions as described herein. The instructions can be stored in a non-transitory, computer-readable storage medium, for example.

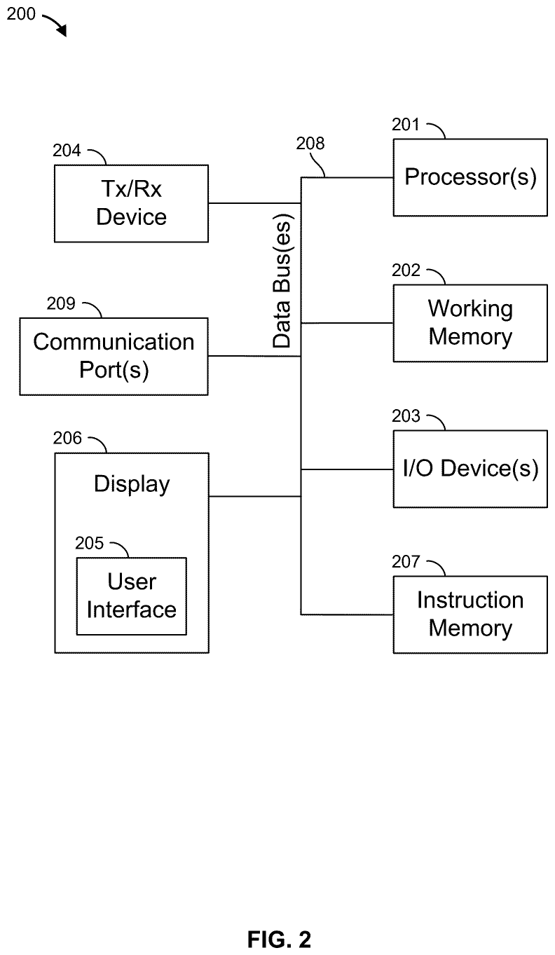

For example, FIG. 2 illustrates a computing device 200 that can be employed by the image processing system 104. Computing device 200 can implement, for example, one or more of the functions of image processing system 104 described herein.

Computing device 200 can include one or more processors 201, working memory 202, one or more input/output devices 203, instruction memory 207, a transceiver 204, one or more communication ports 209, and a display 206, all operatively coupled to one or more data buses 208. Data buses 208 allow for communication among the various devices. Data buses 208 can include wired, or wireless, communication channels.

Processors 201 can include one or more distinct processors, each having one or more cores. Each of the distinct processors can have the same or different structure. Processors 201 can include one or more central processing units (CPUs), one or more graphics processing units (GPUs), application specific integrated circuits (ASICs), digital signal processors (DSPs), and the like.

Processors 201 can be configured to perform a certain function or operation by executing code, stored on instruction memory 207, embodying the function or operation. For example, processors 201 can be configured to perform one or more of any function, method, or operation disclosed herein.

Instruction memory 207 can store instructions that can be accessed (e.g., read) and executed by processors 201. For example, instruction memory 207 can be a non-transitory, computer-readable storage medium such as a read-only memory (ROM), an electrically erasable programmable read-only memory (EEPROM), flash memory, a removable disk, CD-ROM, any non-volatile memory, or any other suitable memory. For example, instruction memory 207 can store instructions that, when executed by one or more processors 201, cause one or more processors 201 to perform one or more of the functions of CT image reconstruction engine 139, PET image reconstruction engine 113, feature extraction engine 142, relative motion displacement engine 144, and display generation engine 146.

Processors 201 can store data to, and read data from, working memory 202. For example, processors 201 can store a working set of instructions to working memory 202, such as instructions loaded from instruction memory 207. Processors 201 can also use working memory 202 to store dynamic data created during the operation of computing device 200. Working memory 202 can be a random access memory (RAM) such as a static random access memory (SRAM) or dynamic random access memory (DRAM), or any other suitable memory.

Input/output devices 203 can include any suitable device that allows for data input or output. For example, input/output devices 203 can include one or more of a keyboard, a touchpad, a mouse, a stylus, a touchscreen, a physical button, a speaker, a microphone, or any other suitable input or output device.

Communication port(s) 207 can include, for example, a serial port such as a universal asynchronous receiver/transmitter (UART) connection, a Universal Serial Bus (USB) connection, or any other suitable communication port or connection. In some examples, communication port(s) 207 allows for the programming of executable instructions in instruction memory 207. In some examples, communication port(s) 207 allow for the transfer (e.g., uploading or downloading) of data, such as CT measurement data 133 and PET measurement data 111.

Display 206 can display user interface 205. User interfaces 205 can enable user interaction with computing device 200. For example, user interface 205 can be a user interface for an application that allows for the viewing of final image volumes 191. In some examples, a user can interact with user interface 205 by engaging input/output devices 203. In some examples, display 206 can be a touchscreen, where user interface 205 is displayed on the touchscreen.

Transceiver 204 allows for communication with a network, such as a Wi-Fi network, an Ethernet network, a cellular network, or any other suitable communication network. For example, if operating in a cellular network, transceiver 204 is configured to allow communications with the cellular network. Processor(s) 201 is operable to receive data from, or send data to, a network via transceiver 204.

Referring back to FIG. 1, CT image reconstruction engine 139 receives CT measurement data 133 (e.g., CT raw data) and processes the CT measurement data 133 to generate reconstructed CT images 137. CT image reconstruction engine 139 can generate reconstructed CT images 137 based on corresponding CT measurement data 133 using any suitable method known in the art. For example, CT image reconstruction engine 139 may apply a backprojection-based algorithm or iterative method to the CT measurement data 133 to generate a reconstructed CT image 137. In addition, PET image reconstruction engine 113 receives PET measurement data 111, and processes the PET measurement data 111 to generate reconstructed PET images 115. For example, the PET image reconstruction engine 113 may apply a iterative MLEM-based algorithm to the PET measurement data 111 to generate a reconstructed PET image 115. PET image reconstruction engine 113 can generate reconstructed PET images 115 based on corresponding PET measurement data 111 using any suitable method known in the art.

Further, feature extraction engine 142 receives CT images 137 and PET images 115, and applies a first trained neural network (e.g., a trained CNN) to the CT images 137 and the PET images 115 to generate joint feature data 143. For instance, the first trained neural network is configured to extract features from the CT images 137 and the PET images 115. As such, the joint feature data 143 may include CT features (e.g., a CT feature map) characterizing features of each CT image 137, and may further include PET features (e.g., a PET feature map) characterizing features of each PET image 115.

As described herein, the first trained neural network may be trained to detect features based on labelled CT images and labelled PET images (e.g., ground truth data) during a training period, and further validated with non-labelled CT images and PET images during a validation period.

In some examples, feature extraction engine 142 includes two neural networks, where one is trained to generate the CT features based on the CT images 137, and the other is trained to generate the PET features based on the PET images 115. In these examples, feature extraction engine 142 may input the CT images 137 and the PET images 115 to the corresponding neural networks, and may combine the output of the neural networks to generate the joint feature data 143.

Further, relative motion displacement engine 144 receives the joint feature data 143 from the feature extraction engine 142, and applies a second trained neural network (e.g., a trained CNN) to the joint feature data 143 to generate displacement data 145 characterizing a displacement between the CT features and PET features received within the joint feature data 143. For instance, the joint feature data 143 may include a 3D vector for each corresponding pixel of the CT features and PET features, where each 3D vector includes a displacement value (e.g., offset value) in each of three directions (e.g., x, y, and z directions of an x, y, z coordinate system). The displacement values may identify a number of pixel positions, for instance.

In some instances, the displacement data 145 characterizes displacements from the CT features to the PET features (e.g., a PET pixel is offset from the corresponding CT pixel in each of the three directions by the displacement values of the corresponding 3D vector). In other instances, the displacement data 145 characterizes displacements from the PET features to the CT features (e.g., a CT pixel is offset from the corresponding PET pixel in each of the three directions by the displacement values of the corresponding 3D vector).

As described herein, the second trained neural network may be trained to detect pixel displacements based on labelled CT features and labelled PET features (e.g., ground truth data) during a training period, and further validated with non-labelled CT images and PET images during a validation period.

Further, relative motion displacement engine 144 may store the displacement data 145 in the data repository 160. In some examples, relative motion displacement engine 144 transmits the displacement data 145 to monitor 162 for display. For instance, monitor 162 may display the corresponding displacement values of the displacement data 145. In some examples, the displacement data 145 is transmitted over a network (e.g., via transceiver 204) to a remote computing device, such as a laptop, smartphone, tablet, or any other suitable computing device. FIG. 4B, for instance, illustrates an image 420 of exemplary displacement values.

As illustrated, display generation engine 146 may receive the displacement data 145 from relative motion displacement engine 144 or, in some examples, from the data repository 160. Based on the displacement data 145, display generation engine 146 may generate display data 147 for display, such as for display on monitor 162. Display data 147 may include a warning message (e.g., icon), a heat map, an image (e.g., alone or superimposed with one or more of the corresponding CT image 137 and PET image 115), or any other suitable data for display that is based on the displacement data 145. In some examples, the display data 147 is transmitted over a network (e.g., via transceiver 204) to a remote computing device, such as a laptop, smartphone, tablet, or any other suitable computing device.

For example, based on the displacement data 145, display generation engine 146 may generate a heat map that represents a 3D displacement magnitude for each of a plurality of portions of the displacement data 145, such as for each corresponding voxel of the displacement data 145. Display generation engine 146 may generate each voxel's 3D displacement magnitude by computing a displacement magnitude value for each pixel of a voxel based on the corresponding displacement values identified within the displacement data 145, and summing the displacement magnitude values for the voxel. Display generation engine 146 may compute the displacement magnitude value for each pixel by determining the magnitude of the pixel's 3D displacement vector. For instance, display generation engine 146 may compute the displacement magnitude value for a pixel according to the Euclidian magnitude of a vector formula below:

$$ℳ = \sqrt{{d}_{{x}^{2}} + {d}_{{y}^{2}} + {x}_{{2}^{2}}} ,$$

where:
   dₓ is the “x” coordinate displacement value;
   d_{y} is the “y” coordinate displacement value; and
   d_{z} is the “z” coordinate displacement value.

Display generation engine 146 may package the computed 3D displacement magnitudes within display data 147, and may transmit the display data 147 to monitor 162 for display. For instance, FIG. 4C illustrates an exemplary heat map 440 that may be generated based on displacement data 145 as described herein.

In some examples, display generation engine 146 may generate display data 147 as an alert to a medical professional. The display data 147 may indicate a warning to the medical professional, for example. For instance, display generation engine 146 may determine whether the displacement data 145 indicates subject movement (e.g., unacceptable subject movement) based on comparing the displacement data 145 to one or more thresholds. If display generation engine 146 determines that the displacement data 145 indicates subject movement, display generation engine 146 may generate display data 147 to indicate a warning or alert.

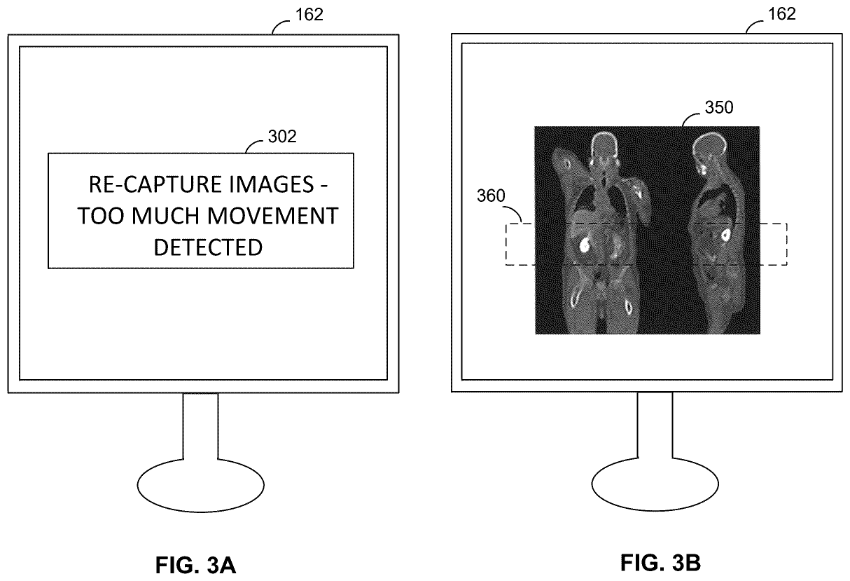

As an example, FIG. 3A illustrates an alert window 302 displayed by monitor 162 and generated based on exemplary display data 147 received from display generation engine 146. The alert window 302 indicates that too much movement has been detected, and that the subject should be re-scanned (e.g., before leaving the imaging room).

To determine whether the displacement data 145 indicates too much movement, display generation engine 146 may compare the computed 3D displacement magnitudes within display data 147 to a threshold, and may determine unacceptable subject movement when any of the computed 3D displacement magnitudes exceed the threshold. As another example, display generation engine 146 may sum the computed 3D displacement magnitudes within display data 147 to determine a total displacement magnitude, and may compare the total displacement magnitude to a threshold. Display generation engine 146 may determine unacceptable subject movement when the total displacement magnitude exceeds the threshold.

In yet other examples, display generation engine 146 may receive from a display, such as monitor 162, selection data characterizing an area of an image, such as any of the CT image 137 or PET image 115. For example, FIG. 3B illustrates a display of a mis-registered PET/CT image 350, as well as a selection box 360. A medical professional may size the selection box 360 (e.g., using an input/output device 203) to select an area of the PET/CT image 350. Display generation engine 146 may receive from the monitor 162 selection data characterizing the selection box 360, and may determine whether one or more of the displacement magnitudes within display data 147 that correspond to pixels within the selection box 360 exceed a corresponding threshold. Display generation engine 146 may determine unacceptable subject movement when the one or more of the displacement magnitudes exceed their corresponding threshold.

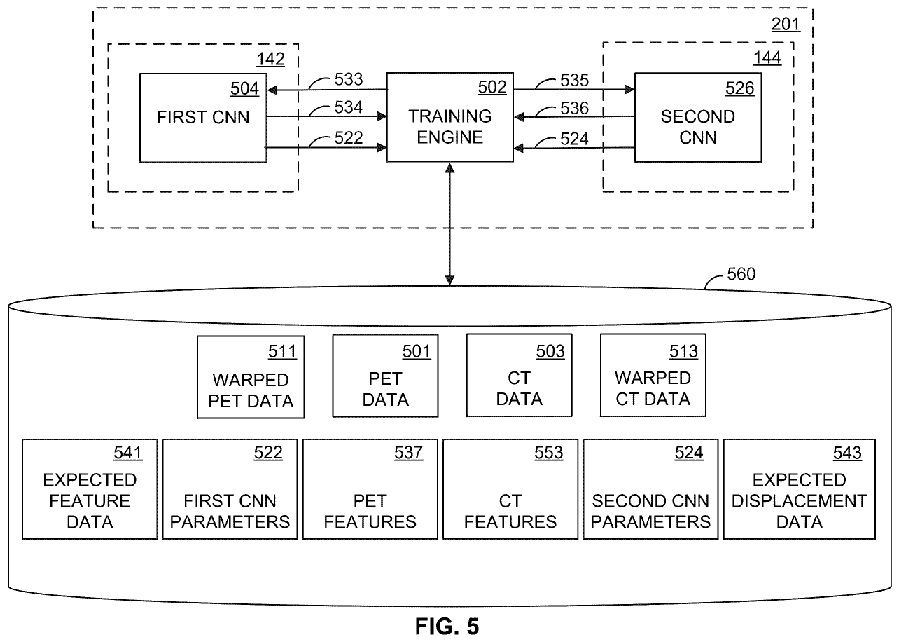

FIG. 5 illustrates a training engine 502 that can train any of the neural networks described herein, such as any of the neural networks described with respect to FIG. 1. Training engine 502 can be implemented by image processing system 104 or computing device 200.

As illustrated, training engine 502 is communicatively coupled to a first CNN 504 a second CNN 526, and a data repository 560. The first CNN 504 may be implemented by feature extraction engine 142, and the second CNN 526 may be implemented by relative motion displacement engine 526. In some examples, the first CNN 504, second CNN 526, and the training engine 502 are implemented by one or more processors 201 executing corresponding instructions.

The data repository 560 includes PET data 501 and CT data 503. Each of the PET data 501 and CT data 503 may be labelled with features (e.g., identifying anatomical regions, tissues, etc.). The PET data 501 may characterize PET images, while the CT data 503 may characterize corresponding CT images. For instance, each PET image may have a corresponding CT image (e.g., the CT image is a scan of a same subject as the corresponding PET image). The data repository 560 also includes PET features 551 and CT features 553. The PET features 551 characterizes features of PET images (e.g., PET images of the PET data 501), and the CT features characterizes features of CT images (e.g., CT images of the CT data 503). Further, the data repository 560 includes expected feature data 541 characterizing features of the images of the PET data 501 and CT data 503, as well as expected displacement data 543 characterizing displacement values between the PET features 551 and the CT features 553.

In some examples, training engine 502 obtains PET data 501, and applies an adjustment to the PET data 501 to generate warped PET data 511. For instance, the training engine 502 may adjust a position of one or more pixels of the PET data 501, and may store the adjusted PET data as warped PET data 511. To adjust the position of pixels, the training engine 502 may copy pixel values (e.g., for each of three dimensions), and write the pixel values to displaced pixel locations. As such, the warped PET data 511 may characterize PET images with displaced (e.g., moved) pixels, thereby generating a PET image this is displaced, at least in part, from a corresponding CT image. Similarly, the training engine 502 may adjust a position of one or more pixels of the CT data 503, and may store the adjusted CT data as warped CT data 513. The warped CT data 513 may characterize CT images with displaced (e.g., moved) pixels, thereby generating a CT image this is displaced, at least in part, from a corresponding PET image.

The training engine 502 may further update the expected feature data 541 and the expected displacement data 543 for the generated images of the warped PET data 511 and warped CT data 513. For instance, the training engine 502 may determine a new location of features based on the displacement used to generate any of the warped PET data 511 and warped CT data 513. Further, and based on the new location of features, the training engine 502 may store values characterizing features and displacements in the expected feature data 541 and the expected displacement data 543, respectively, for any generated warped PET data 511 and warped CT data 513.

Training engine 502 may obtain from data repository 560 PET data 501 and CT data 503, and may generate first training data 533. The first training data 533 may include, for example, labelled 3D PET image vectors and corresponding labelled 3D CT image vectors. In some instances, training engine 502 generates at least portions of the first training data 533 based on one or more of warped PET data 511 and CT data 503, PET data 501 and warped CT data 513, and warped PET data 511 and warped CT data 513. Training engine 502 inputs the first training data 533 to the first CNN 504, and receives from the first CNN 504 corresponding output data 534. The output data 534 may characterize detected features for each of the inputted PET and CT images.

Training engine 502 may receive the output data 534 from the first CNN 504, and may determine whether the first CNN 504 is trained based on the output data 534. For example, training engine 502 may compute a metric value based on the output data 534 and expected feature data 541 characterizing expected feature detections. The metric value may be, for instance, a metric value computed from a loss function, such as computed precision values, computed recall values, a computed AUC value, any ROC curve or PR curve value, or any other suitable metric value. Training engine 502 may determine that the first CNN 504 is trained when the computed metric value is beyond (e.g., above, below) a corresponding threshold.

Training engine 502 may train the first CNN 504 with first training data 533 during a training period, and may further validate the first CNN 504 with additional, non-labelled, first training data 533 during a validation period. To determine whether the first CNN 504 is validated, the training engine 502 may compute a metric value based on the output data 534 generated during the validation period. The training engine 502 may determine that the first CNN 504 is trained when the metric value is beyond a corresponding threshold.

When the first CNN 504 is trained, the training engine 502 may obtain first CNN parameters 522 from the trained first CNN 504. The first CNN parameters 522 may include, for instance, hyperparameters, weights, coefficients, and/or any other values needed to establish the trained first CNN 504. The training engine 502 may store the first CNN parameters 522 in data repository 560. For inference, the feature extraction engine 142 may obtain the first CNN parameters 522 from the data repository 560, and may establish (e.g., execute) the trained first CNN 504 based on the first CNN parameters 522.

In addition, training engine 502 may obtain from data repository 560 PET features 551 and CT features 553, and may generate second training data 535. The second training data 535 may include, for example, labelled PET features and corresponding labelled CT features. In some instances, PET features 551 includes labelled features generated from one or both of PET data 501 and warped PET data 511. In some instances, CT features 553 includes labelled features generated from one or both of CT data 503 and warped CT data 513. Training engine 502 inputs the second training data 535 to the second CNN 526, and receives from the second CNN 526 corresponding output data 536. The output data 536 may characterize displacement values between the PET features 551 and corresponding CT features 553.

Training engine 502 may receive the output data 536 from the second CNN 526, and may determine whether the second CNN 526 is trained based on the output data 536. For example, training engine 502 may compute a metric value based on the output data 536 and expected displacement data 543 characterizing expected displacements. The metric value may be, for instance, a metric value computed from a loss function, such as computed precision values, computed recall values, a computed AUC value, any ROC curve or PR curve value, or any other suitable metric value. Training engine 502 may determine that the second CNN 526 is trained when the computed metric value is beyond (e.g., above, below) a corresponding threshold.

Training engine 502 may train the second CNN 526 with second training data 535 during a training period, and may further validate the second CNN 526 with additional, non-labelled, second training data 535 during a validation period. To determine whether the second CNN 526 is validated, the training engine 502 may compute a metric value based on the output data 536 generated during the validation period. The training engine 502 may determine that the second CNN 526 is trained when the metric value is beyond a corresponding threshold.

When the second CNN 526 is trained, the training engine 502 may obtain second CNN parameters 524 from the trained second CNN 526. The second CNN parameters 524 may include, for instance, hyperparameters, weights, coefficients, and/or any other values needed to establish the trained second CNN 526. The training engine 502 may store the second CNN parameters 524 in data repository 560. For inference, the relative motion displacement engine 144 may obtain the second CNN parameters 524 from the data repository 560, and may establish (e.g., execute) the trained second CNN 526 based on the second CNN parameters 524.

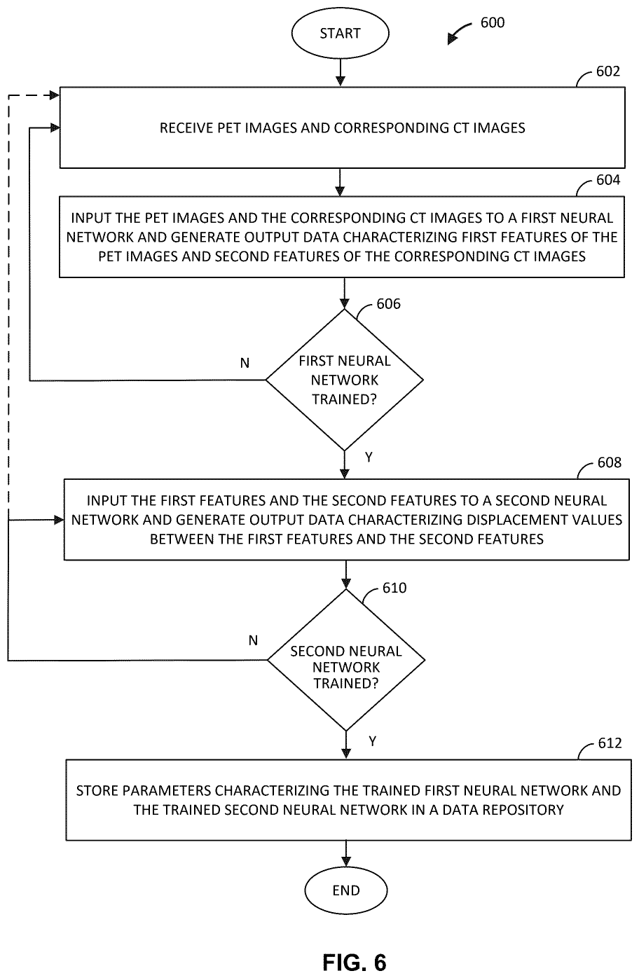

FIG. 6 is a flowchart of an example method 600 to train neural networks, such as the neural networks described with respect to FIG. 1. The method can be performed by one or more computing devices, such as image processing system 104, executing corresponding instructions.

Beginning at block 602, PET images and corresponding CT images are received. For example, image processing system 104 may generate PET images 115 and CT images 137, respectively, from PET measurement data 111 and CT measurement data 133 received from the image scanning system 102. The CT images and PET images may be labelled. For instance, the labels may identify features of each of the CT images and PET images. At block 604, the PET images and CT images may be inputted to a first neural network. Based on inputting the PET images and the CT images to the first neural network, output data is generated. The output data (e.g., joint feature data 143) characterizes first features of the PET images and second features of the CT images.

Proceeding to block 606, a determination is made as to whether the first neural network is trained based on the output data. For example, as described herein, image processing system 104 may compute a metric value based on the output data and expected feature data characterizing expected feature detections. The metric value may be, for instance, a metric value computed from a loss function, such as computed precision values, computed recall values, a computed AUC value, any ROC curve or PR curve value, or any other suitable metric value. Image processing system 104 may determine that the first neural network is trained when the computed metric value is beyond (e.g., above, below) a corresponding threshold.

In some instances, the first trained neural network is trained during a training period, and further validated with non-labelled CT images and non-labelled PET images during a validation period. A metric value may be computed based on output data generated during the validation period, and the first neural network may be considered trained when a metric value is beyond a corresponding threshold. If the first neural network is not sufficiently trained, the method proceeds to back to block 602 to continue training the first neural network. Otherwise, if the first neural network is sufficiently trained, the method proceeds to block 608.

At block 608, the first features and the second features are inputted to a second neural network. Based on inputting the first features and the second features to the second neural network, output data is generated. The output data (e.g., displacement data 145) characterizes displacement values between the first features of the PET images and the second features of the CT images.

Proceeding to block 610, a determination is made as to whether the second neural network is trained based on the output data. For example, as described herein, image processing system 104 may compute a metric value based on the output data and expected displacement data characterizing expected displacement values between the first features and the second features. The metric value may be, for instance, a metric value computed from a loss function, such as computed precision values, computed recall values, a computed AUC value, any ROC curve or PR curve value, or any other suitable metric value. Image processing system 104 may determine that the second neural network is trained when the computed metric value is beyond (e.g., above, below) a corresponding threshold.

In some instances, the second trained neural network is trained during a training period, and further validated with non-labelled displacement data during a validation period. A metric value may be computed based on output data generated during the validation period, and the second neural network may be considered trained when the metric value is beyond a corresponding threshold. If the second neural network is not sufficiently trained, the method proceeds to back to block 608 to continue training the second neural network or, alternatively, to block 602 to continue training the first neural network. Otherwise, if the second neural network is sufficiently trained, the method proceeds to block 612.

At block 612, parameters characterizing the trained first neural network, and the trained second neural network, are stored in a data repository (e.g., data repository 160). The parameters may include, for example, hyperparameters, weights, coefficients, and any other relevant values. The image processing system 104 may establish each of the trained first neural network and the trained second neural network based on the stored parameters.

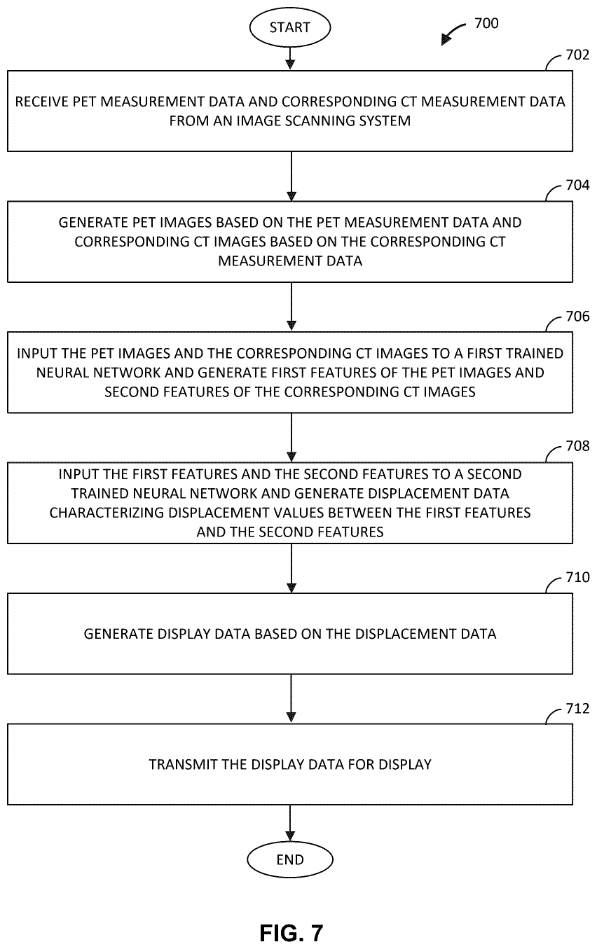

FIG. 7 is a flowchart of an example method 700 to detect subject motion using trained neural networks, such as the trained neural networks described with respect to FIG. 1. The method can be performed by one or more computing devices, such as image processing system 104, executing corresponding instructions.

Beginning at block 702, PET measurement data and CT measurement data is received from an image scanning system. At block 704, PET images are generated based on the PET measurement data, and CT images are generated based on the CT measurement data. For example, as described herein, image processing system 104 may receive PET measurement data 111 and CT measurement data 133 from image scanning system 102, and may generate PET images 115 and CT images 137, respectively, based on the PET measurement data 111 and CT measurement data 133.

At block 706, the PET images and CT images are inputted to a first trained neural network. Based on inputting the PET images and CT images to the first trained neural network, first features for the PET images and second features for the CT images are generated. For example, as described herein, image processing system 104 may input CT images 137 and PET images 115 to a first trained neural network and, in response, the first trained neural network may output joint feature data 143 characterizing CT features and PET features, respectively.

Further, at block 708, the first features and the second features are input to a second trained neural network. Based on inputting the first features and the second features to the second trained neural network, displacement data is generated. The displacement data characterizes displacement values between the first features and the second features. For example, as described herein, the image processing system 104 may input the joint feature data 143 to a second trained neural network. Based on inputting the joint feature data 143 to the second trained neural network, the second trained neural network may output displacement data 145 characterizing displacement values between the CT features and PET features.

Proceeding to block 710, display data is generated based on the displacement data. For instance, the display data may be a warning message (e.g., alert window 302), a heat map (e.g., heat map 440), or an image (e.g., image 420), as described herein. At block 712, the display data is transmitted for display (e.g., display on monitor 162). In some instances, the display data is transmitted over a network (e.g., via transceiver 204) to a remote computing device, such as a laptop, smartphone, tablet, or any other suitable computing device.

The embodiments described herein may employ a deep learning motion estimation framework that includes two trained CNNs. The first trained CNN processes PET/SPECT images and co-modality (e.g., (CT) images to extract features (e.g., common features) from each of the PET/SPECT images and co-modality images. The second trained CNN uses the two sets of generated features to detect relative motion displacement between the two sets of features. A magnitude of the relative motion displacements may be determined, and may be used to display information, such as a heat map, relating to the detected inter-modal movement.

The following is a list of non-limiting illustrative embodiments disclosed herein:

Illustrative Embodiment 1: A computer-implemented method comprising:
receiving positron emission tomography (PET) measurement data and co-modality measurement data from an image scanning system;
generating a PET image based on the PET measurement data and a co-modality image based on the co-modality measurement data;
inputting the PET image and the co-modality image to a first trained neural network and, based on inputting the PET image and the co-modality image to the first trained neural network, generating first features of the PET image and second features of the co-modality image;
inputting the first features and the second features to a second trained neural network and, based on inputting the first output data to the second trained neural network, generating displacement data characterizing a displacement between the first features and the second features; and
generating display data based on the displacement data, and transmitting the display data for display.

Illustrative Embodiment 2: The computer-implemented method of illustrative embodiment 1 wherein the co-modality measurement data is computed tomography (CT) measurement data and the co-modality images are CT images.

Illustrative Embodiment 3: The computer-implemented method of any of illustrative embodiments 1-2 wherein the first trained neural network is a convolutional neural network (CNN).

Illustrative Embodiment 4: The computer-implemented method of any of illustrative embodiments 1-3 wherein the second trained neural network is a convolutional neural network (CNN).

Illustrative Embodiment 5: The computer-implemented method of any of illustrative embodiments 1-4 wherein the first features of the PET images and the second features of the co-modality images include common features.

Illustrative Embodiment 6: The computer-implemented method of any of illustrative embodiments 1-5 wherein the displacement data comprises at least one displacement value for each of a plurality of pixels of the PET image and the co-modality image.

Illustrative Embodiment 7: The computer-implemented method of illustrative embodiment 6 wherein the at least one displacement value for each of the plurality of pixels comprises a first displacement value for a first direction, a second displacement value for a second direction, and a third displacement value for a third direction.

Illustrative Embodiment 8: The computer-implemented method of illustrative embodiment 7 comprising:
determining, for each of the plurality of pixels, a magnitude value based on the first displacement value, the second displacement value, and the third displacement value; and
generating the display data based on the magnitude values.

Illustrative Embodiment 9: The computer-implemented method of illustrative embodiment 8 wherein the display data characterizes a heat map.

Illustrative Embodiment 10: The computer-implemented method of any of illustrative embodiments 1-9 wherein the displacement data comprises displacement values identifying pixel offsets between the PET image and the co-modality image.

Illustrative Embodiment 11: The computer-implemented method of any of illustrative embodiments 1-10 wherein the PET measurement data and the co-modality measurement data are based on corresponding scans of a same subject.

Illustrative Embodiment 12: The computer-implemented method of any of illustrative embodiments 1-11 comprising training the first trained neural network, the training comprising:
inputting labelled PET images and labelled CT images to a neural network and, based on inputting the labelled PET images and the labelled CT images to the neural network, generating output data characterizing PET features and CT features; and
determining the neural network is trained based on the output data.

Illustrative Embodiment 13: The computer-implemented method of illustrative embodiment 12 comprising:
determining at least one metric value based on the output data; and
determining the neural network is trained based on the at least one metric value.

Illustrative Embodiment 14: The computer-implemented method of any of illustrative embodiments 12-13 comprising storing parameters characterizing the first trained neural network in a data repository.

Illustrative Embodiment 15: The computer-implemented method of any of illustrative embodiments 1-14 comprising training the second trained neural network, the training comprising:
inputting labelled PET features and labelled CT features to a neural network and, based on inputting the labelled PET features and the labelled CT features to the neural network, generating output data characterizing displacement values between the labelled PET features and labelled CT features; and
determining the neural network is trained based on the output data.

Illustrative Embodiment 16: The computer-implemented method of illustrative embodiment 15 comprising:
determining at least one metric value based on the output data; and
determining the neural network is trained based on the at least one metric value.

Illustrative Embodiment 17: The computer-implemented method of any of illustrative embodiments 15-16 comprising storing parameters characterizing the first trained neural network in a data repository.

Illustrative Embodiment 18: A non-transitory computer readable medium storing instructions that, when executed by at least one processor, cause the at least one processor to perform operations comprising:
receiving positron emission tomography (PET) measurement data and co-modality measurement data from an image scanning system;
generating a PET image based on the PET measurement data and a co-modality image based on the co-modality measurement data;
inputting the PET image and the co-modality image to a first trained neural network and, based on inputting the PET image and the co-modality image to the first trained neural network, generating first features of the PET image and second features of the co-modality image;
inputting the first features and the second features to a second trained neural network and, based on inputting the first output data to the second trained neural network, generating displacement data characterizing a displacement between the first features and the second features; and
generating display data based on the displacement data, and transmitting the display data for display.

Illustrative Embodiment 19: The non-transitory computer readable medium of illustrative embodiment 18 wherein the co-modality measurement data is computed tomography (CT) measurement data and the co-modality images are CT images.

Illustrative Embodiment 20: The non-transitory computer readable medium of any of illustrative embodiments 18-19 wherein the first trained neural network is a convolutional neural network (CNN).

Illustrative Embodiment 21: The non-transitory computer readable medium of any of illustrative embodiments 18-20 wherein the second trained neural network is a convolutional neural network (CNN).

Illustrative Embodiment 22: The non-transitory computer readable medium of any of illustrative embodiments 18-21 wherein the first features of the PET images and the second features of the co-modality images include common features.

Illustrative Embodiment 23: The non-transitory computer readable medium of any of illustrative embodiments 8-22 wherein the displacement data comprises at least one displacement value for each of a plurality of pixels of the PET image and the co-modality image.

Illustrative Embodiment 24: The non-transitory computer readable medium of illustrative embodiment 23 wherein the at least one displacement value for each of the plurality of pixels comprises a first displacement value for a first direction, a second displacement value for a second direction, and a third displacement value for a third direction.

Illustrative Embodiment 25: The non-transitory computer readable medium of illustrative embodiment 24 wherein the instructions, when executed by the at least one processor, further cause the at least one processor to perform operations comprising:
determining, for each of the plurality of pixels, a magnitude value based on the first displacement value, the second displacement value, and the third displacement value; and
generating the display data based on the magnitude values.

Illustrative Embodiment 26: The non-transitory computer readable medium of illustrative embodiment 25 wherein the display data characterizes a heat map.

Illustrative Embodiment 27: The non-transitory computer readable medium of any of illustrative embodiments 18-26 wherein the displacement data comprises displacement values identifying pixel offsets between the PET image and the co-modality image.

Illustrative Embodiment 28: The non-transitory computer readable medium of any of illustrative embodiments 18-27 wherein the PET measurement data and the co-modality measurement data are based on corresponding scans of a same subject.

Illustrative Embodiment 29: The non-transitory computer readable medium of any of illustrative embodiments 18-28 wherein the instructions, when executed by the at least one processor, further cause the at least one processor to perform operations comprising training the first trained neural network, the training comprising:
inputting labelled PET images and labelled CT images to a neural network and, based on inputting the labelled PET images and the labelled CT images to the neural network, generating output data characterizing PET features and CT features; and
determining the neural network is trained based on the output data.

Illustrative Embodiment 30: The non-transitory computer readable medium of illustrative embodiment 29 wherein the instructions, when executed by the at least one processor, further cause the at least one processor to perform operations comprising:
determining at least one metric value based on the output data; and
determining the neural network is trained based on the at least one metric value.

Illustrative Embodiment 31: The non-transitory computer readable medium of any of illustrative embodiments 29-30 wherein the instructions, when executed by the at least one processor, further cause the at least one processor to perform operations comprising storing parameters characterizing the first trained neural network in a data repository.

Illustrative Embodiment 32: The non-transitory computer readable medium of any of illustrative embodiments 18-31 wherein the instructions, when executed by the at least one processor, further cause the at least one processor to perform operations comprising training the second trained neural network, the training comprising:
inputting labelled PET features and labelled CT features to a neural network and, based on inputting the labelled PET features and the labelled CT features to the neural network, generating output data characterizing displacement values between the labelled PET features and labelled CT features; and
determining the neural network is trained based on the output data.

Illustrative Embodiment 33: The non-transitory computer readable medium of illustrative embodiment 32 wherein the instructions, when executed by the at least one processor, further cause the at least one processor to perform operations comprising:
determining at least one metric value based on the output data; and
determining the neural network is trained based on the at least one metric value.

Illustrative Embodiment 34: The non-transitory computer readable medium of any of illustrative embodiments 32-33 wherein the instructions, when executed by the at least one processor, further cause the at least one processor to perform operations comprising storing parameters characterizing the first trained neural network in a data repository.

Illustrative Embodiment 35: A system comprising:
a memory device storing instructions; and
at least one processor communicatively coupled the memory device, the at least one processor configured to execute the instructions to:
   receive positron emission tomography (PET) measurement data and co-modality measurement data from an image scanning system;
   generate a PET image based on the PET measurement data and a co-modality image based on the co-modality measurement data;
   input the PET image and the co-modality image to a first trained neural network and, based on inputting the PET image and the co-modality image to the first trained neural network, generate first features of the PET image and second features of the co-modality image;
   input the first features and the second features to a second trained neural network and, based on inputting the first output data to the second trained neural network, generate displacement data characterizing a displacement between the first features and the second features; and
   generate display data based on the displacement data, and transmitting the display data for display.

Illustrative Embodiment 36: The system of illustrative embodiment 35 wherein the co-modality measurement data is computed tomography (CT) measurement data and the co-modality images are CT images.

Illustrative Embodiment 37: The system of any of illustrative embodiments 35-36 wherein the first trained neural network is a convolutional neural network (CNN).

Illustrative Embodiment 38: The system of any of illustrative embodiments 35-37 wherein the second trained neural network is a convolutional neural network (CNN).

Illustrative Embodiment 39: The system of any of illustrative embodiments 35-38 wherein the first features of the PET images and the second features of the co-modality images include common features.

Illustrative Embodiment 40: The system of any of illustrative embodiments 35-39 wherein the displacement data comprises at least one displacement value for each of a plurality of pixels of the PET image and the co-modality image.

Illustrative Embodiment 41: The system of illustrative embodiment 40 wherein the at least one displacement value for each of the plurality of pixels comprises a first displacement value for a first direction, a second displacement value for a second direction, and a third displacement value for a third direction.

Illustrative Embodiment 42: The system of illustrative embodiment 41 wherein the at least one processor is configured to execute the instructions to:
determine, for each of the plurality of pixels, a magnitude value based on the first displacement value, the second displacement value, and the third displacement value; and
generate the display data based on the magnitude values.

Illustrative Embodiment 43: The system of illustrative embodiment 42 wherein the display data characterizes a heat map.

Illustrative Embodiment 44: The system of any of illustrative embodiments 35-43 wherein the displacement data comprises displacement values identifying pixel offsets between the PET image and the co-modality image.

Illustrative Embodiment 45: The system of any of illustrative embodiments 35-44 wherein the PET measurement data and the co-modality measurement data are based on corresponding scans of a same subject.

Illustrative Embodiment 46: The system of any of illustrative embodiments 35-45 wherein the at least one processor, to train the first trained neural network, is configured to execute the instructions to:
input labelled PET images and labelled CT images to a neural network and, based on inputting the labelled PET images and the labelled CT images to the neural network, generate output data characterizing PET features and CT features; and
determine the neural network is trained based on the output data.

Illustrative Embodiment 47: The system of illustrative embodiment 46 wherein the at least one processor is configured to execute the instructions to:
determine at least one metric value based on the output data; and
determine the neural network is trained based on the at least one metric value.

Illustrative Embodiment 48: The system of any of illustrative embodiments 46-47 wherein the at least one processor is configured to execute the instructions to store parameters characterizing the first trained neural network in a data repository.

Illustrative Embodiment 49: The system of any of illustrative embodiments 35-48 wherein the at least one processor, to train the second trained neural network, is configured to execute the instructions to:
input labelled PET features and labelled CT features to a neural network and, based on inputting the labelled PET features and the labelled CT features to the neural network, generate output data characterizing displacement values between the labelled PET features and labelled CT features; and
determine the neural network is trained based on the output data.

Illustrative Embodiment 50: The system of illustrative embodiment 49 wherein the at least one processor is configured to execute the instructions to:
determine at least one metric value based on the output data; and
determine the neural network is trained based on the at least one metric value.

Illustrative Embodiment 51: The system of any of illustrative embodiments 49-50 wherein the at least one processor is configured to execute the instructions to store parameters characterizing the first trained neural network in a data repository.

Illustrative Embodiment 52: A system comprising:
a means for receiving positron emission tomography (PET) measurement data and co-modality measurement data from an image scanning system;
a means for generating a PET image based on the PET measurement data and a co-modality image based on the co-modality measurement data;
a means for inputting the PET image and the co-modality image to a first trained neural network and, based on inputting the PET image and the co-modality image to the first trained neural network, generating first features of the PET image and second features of the co-modality image;
a means for inputting the first features and the second features to a second trained neural network and, based on inputting the first output data to the second trained neural network, generating displacement data characterizing a displacement between the first features and the second features; and
a means for generating display data based on the displacement data, and transmitting the display data for display.

Illustrative Embodiment 53: The system of illustrative embodiment 52 wherein the co-modality measurement data is computed tomography (CT) measurement data and the co-modality images are CT images.

Illustrative Embodiment 54: The system of any of illustrative embodiments 52-53 wherein the first trained neural network is a convolutional neural network (CNN).

Illustrative Embodiment 55: The system of any of illustrative embodiments 52-54 wherein the second trained neural network is a convolutional neural network (CNN).

Illustrative Embodiment 56: The system of any of illustrative embodiments 52-55 wherein the first features of the PET images and the second features of the co-modality images include common features.

Illustrative Embodiment 57: The system of any of illustrative embodiments 52-56 wherein the displacement data comprises at least one displacement value for each of a plurality of pixels of the PET image and the co-modality image.

Illustrative Embodiment 58: The system of illustrative embodiment 57 wherein the at least one displacement value for each of the plurality of pixels comprises a first displacement value for a first direction, a second displacement value for a second direction, and a third displacement value for a third direction.

Illustrative Embodiment 59: The system of illustrative embodiment 58 comprising:
a means for determining, for each of the plurality of pixels, a magnitude value based on the first displacement value, the second displacement value, and the third displacement value;
and a means for generating the display data based on the magnitude values.

Illustrative Embodiment 60: The system of illustrative embodiment 59 wherein the display data characterizes a heat map.

Illustrative Embodiment 61: The system of any of illustrative embodiments 52-60 wherein the displacement data comprises displacement values identifying pixel offsets between the PET image and the co-modality image.

Illustrative Embodiment 62: The system of any of illustrative embodiments 52-61 wherein the PET measurement data and the co-modality measurement data are based on corresponding scans of a same subject.

Illustrative Embodiment 63: The system of any of illustrative embodiments 52-62 comprising a means for training the first trained neural network, the means for training comprising:
a means for inputting labelled PET images and labelled CT images to a neural network and, based on inputting the labelled PET images and the labelled CT images to the neural network, generating output data characterizing PET features and CT features; and
a means for determining the neural network is trained based on the output data.

Illustrative Embodiment 64: The system of illustrative embodiment 63 comprising:
a means for determining at least one metric value based on the output data; and
a means for determining the neural network is trained based on the at least one metric value.

Illustrative Embodiment 65: The system of any of illustrative embodiments 63-64 comprising a means for storing parameters characterizing the first trained neural network in a data repository.

Illustrative Embodiment 66: The system of any of illustrative embodiments 52-65 comprising a means for training the second trained neural network, the means for training comprising:

a means for inputting labelled PET features and labelled CT features to a neural network and, based on inputting the labelled PET features and the labelled CT features to the neural network, generating output data characterizing displacement values between the labelled PET features and labelled CT features; and
a means for determining the neural network is trained based on the output data.

Illustrative Embodiment 67: The system of illustrative embodiment 66 comprising:
a means for determining at least one metric value based on the output data; and
a means for determining the neural network is trained based on the at least one metric value.

Illustrative Embodiment 68: The system of any of illustrative embodiments 66-67 comprising a means for storing parameters characterizing the first trained neural network in a data repository.

The apparatuses and processes are not limited to the specific embodiments described herein. In addition, components of each apparatus and each process can be practiced independent and separate from other components and processes described herein.

The previous description of embodiments is provided to enable any person skilled in the art to practice the disclosure. The various modifications to these embodiments will be readily apparent to those skilled in the art, and the generic principles defined herein can be applied to other embodiments without the use of inventive faculty. The present disclosure is not intended to be limited to the embodiments shown herein, but is to be accorded the widest scope consistent with the principles and novel features disclosed herein.

What is claimed is:

1. A computer-implemented method comprising:
receiving positron emission tomography (PET) measurement data and co-modality measurement data from an image scanning system;
generating a PET image based on the PET measurement data and a co-modality image based on the co-modality measurement data;
inputting the PET image and the co-modality image to a first trained neural network and, based on inputting the PET image and the co-modality image to the first trained neural network, generating first features of the PET image and second features of the co-modality image;
inputting the first features and the second features to a second trained neural network and, based on inputting the first output data to the second trained neural network, generating displacement data characterizing a displacement between the first features and the second features; and
generating display data based on the displacement data, and transmitting the display data for display.

2. The computer-implemented method of claim 1 wherein the co-modality measurement data is computed tomography (CT) measurement data and the co-modality images are CT images.

3. The computer-implemented method of claim 1 wherein the first trained neural network is a convolutional neural network (CNN).

4. The computer-implemented method of claim 1 wherein the second trained neural network is a convolutional neural network (CNN).

5. The computer-implemented method of claim 1 wherein the first features of the PET images and the second features of the co-modality images include common features.

6. The computer-implemented method of claim 1 wherein the displacement data comprises at least one displacement value for each of a plurality of pixels of the PET image and the co-modality image.

7. The computer-implemented method of claim 6 wherein the at least one displacement value for each of the plurality of pixels comprises a first displacement value for a first direction, a second displacement value for a second direction, and a third displacement value for a third direction.

8. The computer-implemented method of claim 7 comprising:
determining, for each of the plurality of pixels, a magnitude value based on the first displacement value, the second displacement value, and the third displacement value; and
generating the display data based on the magnitude values.

9. The computer-implemented method of claim 8 wherein the display data characterizes a heat map.

10. The computer-implemented method of claim 1 wherein the displacement data comprises displacement values identifying pixel offsets between the PET image and the co-modality image.

11. The computer-implemented method of claim 1 wherein the PET measurement data and the co-modality measurement data are based on corresponding scans of a same subject.

12. The computer-implemented method of claim 1 comprising training the first trained neural network, the training comprising:
inputting labelled PET images and labelled CT images to a neural network and, based on inputting the labelled PET images and the labelled CT images to the neural network, generating output data characterizing PET features and CT features; and
determining the neural network is trained based on the output data.

13. The computer-implemented method of claim 12 comprising:
determining at least one metric value based on the output data; and
determining the neural network is trained based on the at least one metric value.

14. The computer-implemented method of claim 12 comprising storing parameters characterizing the first trained neural network in a data repository.

15. The computer-implemented method of claim 1 comprising training the second trained neural network, the training comprising:
inputting labelled PET features and labelled CT features to a neural network and, based on inputting the labelled PET features and the labelled CT features to the neural network, generating output data characterizing displacement values between the labelled PET features and labelled CT features; and
determining the neural network is trained based on the output data.

16. The computer-implemented method of claim 15 comprising:
determining at least one metric value based on the output data; and
determining the neural network is trained based on the at least one metric value.

17. The computer-implemented method of claim 15 comprising storing parameters characterizing the first trained neural network in a data repository.

18. A non-transitory computer readable medium storing instructions that, when executed by at least one processor, cause the at least one processor to perform operations comprising:
receiving positron emission tomography (PET) measurement data and co-modality measurement data from an image scanning system;
generating a PET image based on the PET measurement data and a co-modality image based on the co-modality measurement data;
inputting the PET image and the co-modality image to a first trained neural network and, based on inputting the PET image and the co-modality image to the first trained neural network, generating first features of the PET image and second features of the co-modality image;
inputting the first features and the second features to a second trained neural network and, based on inputting the first output data to the second trained neural network, generating displacement data characterizing a displacement between the first features and the second features; and
generating display data based on the displacement data, and transmitting the display data for display.

19. The non-transitory computer readable medium of claim 18 wherein the co-modality measurement data is computed tomography (CT) measurement data and the co-modality images are CT images.

20. A system comprising:
a memory device storing instructions; and
at least one processor communicatively coupled the memory device, the at least one processor configured to execute the instructions to:
receive positron emission tomography (PET) measurement data and co-modality measurement data from an image scanning system;
generate a PET image based on the PET measurement data and a co-modality image based on the co-modality measurement data;
input the PET image and the co-modality image to a first trained neural network and, based on inputting the PET image and the co-modality image to the first trained neural network, generate first features of the PET image and second features of the co-modality image;
input the first features and the second features to a second trained neural network and, based on inputting the first output data to the second trained neural network, generate displacement data characterizing a displacement between the first features and the second features; and
generate display data based on the displacement data, and transmitting the display data for display.
